(19) United States
(12) Patent Application Publication
(10) Pub. No.: US 2025/0065145 A1
(43) Pub. Date: Feb. 27, 2025

(54) LIGHT THERAPY DEVICE ATTACHABLE TO AN ARM OF AN EYEGLASS FRAME

(71) Applicant: TSUBOTA LABORATORY INC., Tokyo (JP)

(72) Inventors: Alexandre R. Tumlinson, South San Francisco, CA (US); Dennis Gorshteyn, South San Francisco, CA (US); Kyle Gilpin, South San Francisco, CA (US); Dimitri Azar, South San Francisco, CA (US)

(21) Appl. No.: 18/797,365

(22) Filed: Aug. 7, 2024

Related U.S. Application Data
(60) Provisional application No. 63/534,111, filed on Aug. 22, 2023.

(51) Int. Cl. A61N 5/06

(52) U.S. Cl. CPC A61N 5/06; A61N 2005/0626; A61N 2005/0632; A61N 2005/0642; A61N 2005/0648; A61N 2005/0661; A61N 2005/0663; A61N 2005/0664

(57) ABSTRACT

A device (including a head portion, a neck portion, and a body portion) can be removably attached to an arm of an eyeglass frame near the hinge to deliver light therapy to a user's eye. The head portion can include a light source that provides the light therapy to the user's eye. The light therapy can include one or more parameters (e.g., dose, time, etc.) and may be controlled based on the position of the light source. The neck portion can be flexible and moveable to orient the light source towards the eye. The body portion can include a controller, at least one sensor, a power source, and the like. The device can include an attachment mechanism to removably attach the device to the arm of the eyeglass frame.

This nonprovisional patent application claims the benefit of the earlier filing date of U.S. provisional application No. 63/534,111 filed 22 Aug. 2023.

### TECHNICAL FIELD

The present disclosure relates to light therapy devices, and, more specifically, to a light therapy device that is removably attachable to an arm of an eyeglass frame and fits between the arm and a user's face to treat one or more ocular conditions and/or neurological conditions.

### BACKGROUND

Light therapy can be used to treat a number of conditions related to the eyes. Traditional light therapy devices are configured as stand-alone tabletop devices/lamps or as wearable eyeglasses or goggle-like devices. Many existing light therapy devices are bulky, breakable, expensive, and include an unsophisticated user interface. One example use where light therapy has been shown to be particularly effective is stopping or delaying the progression of myopia in children. However, children tend to break traditional light therapy devices at a high rate and often refuse to wear bulky and unstylish devices. Moreover, traditional light therapy devices require the child to remain relatively still for a time when the therapy is administered for effective treatment, but convincing the child to effectively use a light therapy device for an appropriate treatment time can be difficult.

### SUMMARY

Described herein is a light therapy device that is attachable to the inside of an arm of an eyeglass frame. The light therapy device can be used on any number of eyeglasses frames and removed and reattached a number of times. The eyeglass frame can be part of a commercially-available eyeglasses with prescription or non-prescription lenses or no lenses at all, for example.

In one aspect, the present disclosure includes a system (including at least a portion of a device configured to attach to an inner side of an arm of an eyeglass frame proximal to a hinge of the eyeglass frame). The device includes a head portion that includes a light source configured to provide light therapy to an eye of a user of the system; a flexible neck portion that can be bendable to orient the light source towards the eye; and a body portion. The body portion includes a controller configured to control at least one parameter related to providing the light therapy, at least one sensor configured to detect a variable related to the wearing of the device and/or the light therapy, a rechargeable power source, and an attachment mechanism configured to removably attach the system to an inner side of an arm of a glasses frame near a temple of the user.

In another aspect, the present disclosure includes a device configured to attach to an inner side of an arm of an eyeglass frame proximal to a hinge of the eyeglass frame. The device includes a substrate with at least one flexible section configured to form a tube around a battery. The device also includes a light source coupled to the substrate and configured to emit light into an eye of a user wearing the eyeglass frame. A flexible housing of the device can be configured to hold the flexible section of the substrate around the battery and aim the light source toward the eye. An elastic band can be configured to attach the device to the eyeglass frame.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is an illustration showing an example implementation of a light therapy device that is removably attachable to an arm of an eyeglass frame and fits between the arm and a user's face near a hinge of the eyeglass frame to treat one or more ocular conditions and/or neurological conditions;

FIG. 2 is a block diagram showing the elements of the light therapy device of FIG. 1 in greater detail;

FIG. 3 is an image of an example of the light therapy device of FIG. 1 implemented on an eyeglass frame;

FIG. 4 is another block diagram showing another example implementation of another light therapy device that is removably attachable to an arm of an eyeglass frame and fits between the arm and a user's face to treat one or more ocular conditions and/or neurological conditions;

FIG. 5A shows an example diagram and FIG. 5B shows an image of the light therapy device of FIG. 4;

FIG. 6 shows the illustration of FIG. 1 with cut lines -BB- and -AA-;

FIG. 7A shows a cut view of the light therapy device along cut line -BB- and FIG. 7B shows a cut view along cut line -AA- of FIG. 6;

FIG. 8A and FIG. 8B show the effect of a refractive surface on the light source of FIGS. 1 and 4;

FIG. 9 is a process flow diagram of an example method for providing light therapy using the light therapy device attached to an inner side of an arm of an eyeglass frame near a hinge of the eyeglass frame;

FIG. 10 is a process flow diagram of an example method for determining whether to deliver light therapy to a user; and

FIG. 11 is a process flow diagram of an example method for providing additional functionality to incentivize a user to use and not to break the light therapy device.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an,” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “light therapy device” refers to a device that can deliver an amount of light into a patient's eye(s) to treat an ocular condition and/or neurological condition. In some instances, the amount of light can be delivered according to a prescription that sets forth the wavelength(s), the dosage, the pulse length, the intensity, the frequency of delivery, the time of delivery, etc. As an example, the prescription may depend on the type of ocular condition and/or neurological condition and/or the severity of the ocular condition and/or neurological condition.

As used herein, the term “ocular condition” refers to a disease, ailment, symptom, or disorder that affects or involves one or more eye, one or more of the parts or regions of the eye, and/or a tissue near the eye. Non-limiting examples of ocular conditions include refractive errors, glaucoma, dry eye, myopia, presbyopia, amblyopia, cataracts, retinopathy, macular degeneration, and the like.

As used herein, the term “neurological condition” refers to a disease, ailment, symptom, or disorder that affects or involves at least a portion of the nervous system (e.g., at least a portion of the brain, at least a portion of the spinal cord, one or more peripheral nerves, one or more glial cells, etc.).

As used herein, the term “eyeglasses” has a similar meaning to the terms “spectacle” and “glasses” and refers to one or more lenses mounted in a frame that holds the one or more lenses in front of a person's eye or eyes and has at least one arm, typically two arms, that each extend over an ear of a wearer. Eyeglasses can be used as at least one of glasses for correcting or treating defective eyesight (e.g., near-sightedness, far-sightedness, astigmatism, or the like), sunglasses, safety glasses, and/or glasses used for merely aesthetic purposes.

As used herein, the term “frame” refers to a device and/or mechanism that is designed to hold the one or more lens in a proper position on a person's head such that the one or more lens is in front of the person's eye(s). Frames exist in a variety of styles, sizes, materials, shapes, and colors. Typically, the frame includes at least a bridge over the nose, rims around at least a portion of each of the one or more lens (typically two lenses) and holding the one or more lens to the frame, and hinged arms (or temples/temple pieces) that when in use extend out from a lateral portion of the rims to temple tips that rest over and/or around a portion of the user's ears.

As used herein, the terms “patient,” “subject,” “person,” “user,” “wearer,” and the like can be used interchangeably and synonymously to refer to an animal (e.g., a human) in need of light therapy who can wear eyeglasses.

### II. Overview

Exposing one or more of a patient's eyes to supplementary, therapeutic light of various spectra may be used as a treatment for one or more ocular conditions and/or neurological conditions. The treatment may be related one or more of the eyes and/or at least a portion of the nervous system. For example, UV light and red light have been used to prevent the progression of and/or treat myopia, bright white light has been used to treat seasonal effective disorder, 40 Hz modulated optical (with or without auditory) stimulus has been shown to promote the metabolism and clearance of destructive proteins associated with aging in the brain and eye, and the like. Current light therapy devices are often bulky, breakable, and expensive, such as standalone tabletop devices/lamps, and/or dedicated wearable devices that are glasses/goggle-like. Children have been documented as receiving benefits from light therapy in the field of myopia prevention, for example, but often have difficulty in complying with treatments (e.g., keeping eye(s) in focus of the therapy for the time), refusing to wear additional headgear, and/or breaking devices. A compact, durable, and low-cost attachment for existing eyeglasses can encourage wider acceptance of light therapy and improve compliance with requirements of light therapy.

To improve the usage of light therapy, a light therapy device has been designed that is removably attachable to an arm of an eyeglass frame and fits between the arm and a user's face (e.g., on an inner side of the arm). Eyeglasses are generally a practical device (e.g., for vision correction and/or safety) but are also a fashion item. It is desirable that the light treatment device be low profile so as to not interrupt the aesthetic of the eyeglasses themselves. Positioning a light therapy device on the inner side of an arm also makes it more difficult for user (e.g., a child) to break the device and for the light therapy to be unobtrusively provided to the eye(s). The light therapy device can be outside the user's direct line of vision near the hinge of the frame where the arm meets the front plate that holds the lens of the eyeglasses (e.g., 5 mm to 2 cm away from the hinge on the arm). The light can be directable toward at least a portion of the eye, for example, so the light only hits the part of the eye intended for treatment. To make it even less likely that children will use and not break the light therapy devices, some of the light therapy devices (and/or associated software) can provide additional functionality such as entertainment functions to incentivize a child to wear glasses with the device attached and receive treatments.

### III. Systems

A user's visual system and/or nervous system can experience numerous benefits by exposing at least one of the user's eyes to supplementary, therapeutic light (e.g., in addition to natural light). The benefits of the therapeutic light can include treatment and/or symptom relief for one or more ocular conditions and/or neurological conditions. Various ocular conditions and/or neurological conditions can be treated through the use of therapeutic light depending on at least the spectra of the light, the intensity of the light, duration of the light, and the location(s) the light is directed toward in and/or around the eye. A portion of the nervous system can also be affected by therapeutic light treatments of the eye. One example of an ocular condition that can be treated with therapeutic light is myopia. Myopia (also referred to as nearsightedness via elongation) is a common progressive vision condition in which objects that are near are seen clearly, but objects farther away are blurry. Specifically, children have seen benefits from light based myopia treatments, including the application of ultraviolet (UV) and red light, to prevent the progression of and/or to treat myopia. Other optical conditions can also be treated with therapeutic light, for example (the following examples are not meant to be limiting): bright white light has been used to treat seasonal effective disorder, 40 Hz modulated optical (and/or auditory) stimulus has been shown to promote the metabolism and clearance of destructive proteins associated with aging in the brain and eye, and the like.

Current devices (e.g., standalone tabletop devices/lamps, and/or dedicated wearable devices that are glasses/goggle-like) designed to deliver the supplementary, therapeutic light applied for the time to the user's eyes are often bulky, breakable, and expensive. Children (and other users) often have trouble complying with treatments using current devices (e.g., keeping eye(s) in focus of the therapy for the time, refusing to wear additional headgear, and/or damaging devices).

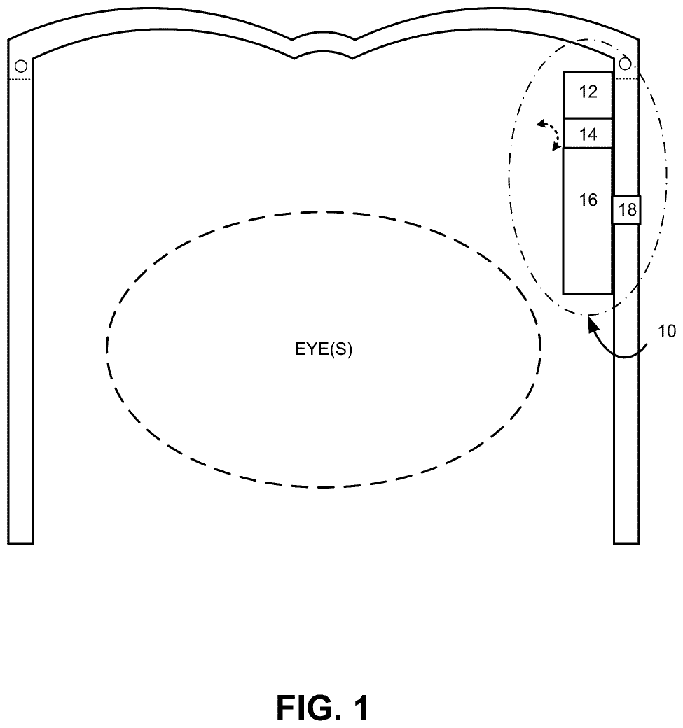

As shown in FIG. 1, a compact, durable, and low-cost attachment for existing eyeglasses can encourage wider acceptance of light therapy and improve user compliance with requirements of light therapy. The attachment can be employed as a system that is also referred to as light therapy device 10 (however, depiction as a device is only for simplicity of explanation; the device is just one example implementation of the system). The light therapy device 10 can be attached to a user's eyeglasses in such a manner as to be able to direct light towards at least one of the eyes of the user. The light therapy device 10 can also be attached to the user's eyeglasses in a manner that can be at least fairly unobtrusive to the user and people looking at the user. The light therapy device 10 can include a head portion 12, a flexible neck portion 14, a body portion 16, and an attachment mechanism 18. The head portion 12 can include a light source configured to provide light therapy to an eye of the user of the system. In some instances, the light used for the light therapy may be concentrated by a refractive surface (e.g., that may be substantially spherical) between the light source and the eye. The flexible neck portion 14 (illustrated as able to bend with a dashed arrow) can be bendable in at least one direction to orient the light source in the head portion 12 towards the eye (or in any desired direction). For example, the light source can be arranged so the light from the light source only (or predominantly) hits a part of the eye intended for treatment (e.g., light concentrated on the pupil of the eye). Additionally, or alternatively, the light can be transscleral illumination. As another example (not illustrated), the light source can be directable towards the lenses of the eyeglasses and the light can be reflected off the lenses of the eyeglasses and into the eye so the light therapy device need not access the hinge area. As a further example (not illustrated), the light source and be directable outwards from the eyeglasses to apply the light via reflection off a work surface through the lenses and back to the user's eye rather than directly into the user's eye. The body portion 16 can include electrical and structural components, described below in further detail, for providing the light therapy as well as the attachment mechanism 18. The attachment mechanism 18 can be part of the body portion 16, as shown, or can be separate from the body portion. The attachment mechanism 18 can be removably attachable to the glasses frame. The light therapy device 10, in some examples, can have a volume less than or equal to 2 cm³ (e.g., vertical orientation less than 10 mm, dimension along the arm of the eyeglasses less than 30 mm).

The light therapy device 10 (or at least a portion of the light therapy device 10) can be removably attachable to an arm of an eyeglass frame (e.g., using the attachment mechanism 18) to provide light therapy (e.g., light having one or more wavelengths, time parameters, intensity parameters, etc.) to at least one eye of a user wearing the eyeglasses for a time. Eyeglasses often serve a practical purpose (e.g., vision correction and/or safety), but can additionally and/or alternatively serve an aesthetic purpose (e.g., as a fashionable accessory). Many users, of any age but especially children and adolescents, may choose to wear eyeglasses and/or light therapy devices in public based on aesthetics rather than treatment suggestions and/or requirements (e.g., bullying, perception by peers, etc.). Therefore, the light therapy device 10 can be designed to have an aesthetically pleasing appearance and/or a low profile (e.g., compared to the frames of the eyeglasses) so as to not interrupt the aesthetic of the eyeglasses themselves. Notably, the light therapy device 10 can be attachable to multiple eyeglasses with arms of a variety of widths, lengths, thicknesses, and materials. The same light therapy device 10 can be used for multiple different eyeglasses (e.g., as the user gets new glasses the device can be compatible with each new pair) and/or used multiple times on the same eyeglasses (e.g., the device can be taken on and off the eyeglasses at will). For example, the light therapy device 10 can be designed to be a size that is smaller than the arm of the smallest possible eyeglasses (e.g., as previously noted, less than 10 mm in total length from one side of the head portion 12 to the furthest side of the body portion 16). The light therapy device 10 can be attachable to the arm of the eyeglasses frame, with the head portion 12 near the hinge of the glasses but outside the majority of the user's line of vision (e.g., 5 mm to 2 cm away from the hinge on the arm to avoid mechanical interference with the hinge or the user's face). The light therapy device 10 can be sized to be able to remain attached to the arm of the eyeglasses frame when the eyeglasses are folded (at the hinge) and not in use or can be removed. Since the hinge is in a variety of places on different eyeglasses, in another example (not illustrated), the light therapy device may attach to the rims, top bar, bridge, and/or end pieces of the eyeglasses.

The light therapy device 10 can be shaped and sized to fit entirely between the arm of the eyeglasses and the user's face. In addition to aesthetic purposes this positioning can make it more difficult for user (e.g., a child) to break the device and can allow the light therapy to be unobtrusively provided to the eye(s). However, it should be noted that the light therapy device 10 can be shaped and sized to fit almost entirely between the frame of the eyeglasses, but with at least a portion extending beyond the frame and/or away from the user's face. As another alternative, a portion of the light therapy device 10 can extend around a portion of the frame of the eyeglasses (e.g., to display a logo, a stylized design, or other visible marking on the outside of the frame while providing more space for electronic components that will not interfere with the user's face). Although the light therapy device 10 being shaped and sized to fit entirely between the arm and the user's face is described throughout, it will be understood that any of the distinctive designs can be used instead.

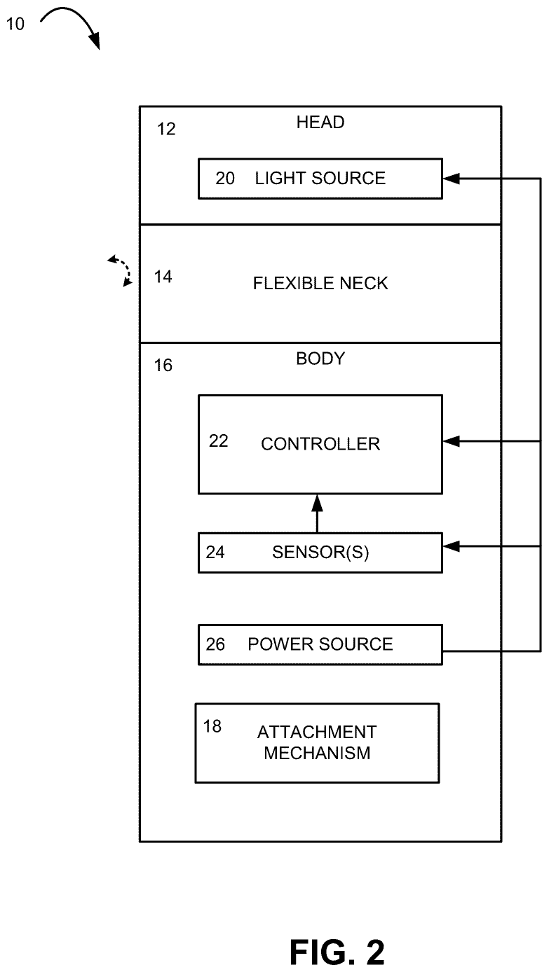

The elements of the light therapy device 10 of FIG. 1 are shown in greater detail in FIG. 2. As noted, the light therapy device 10 can include at least a head portion 12, a flexible neck portion 14, and a body portion 16 with attachment mechanism 18. At least a portion of the head portion 12, the flexible neck portion 14, and/or the body portion 16 can be embodied on and/or as a part of one or more electrical connection substrate (that may have at least one flexible portion and/or at least one non-flexible portion).

The head portion 12 can include the light source 20 that can deliver the one or more doses of light therapy to the user's eye. The head portion can also include a circuit board, which may be a flexible circuit board (or portion of a circuit board) or a partially flexible circuit board (or portion of a circuit board), connecting the light source 20 with one or more electrical and/or computing components of the body portion 14. The head portion 12 with the light source 20 can be positioned near the hinge of the eyeglasses (e.g., on the arm closer to the front of the eyeglasses than the ear. For example, the head portion 12 can be between 5 mm and 2 cm away from the hinge on the arm to avoid mechanical interference with the hinge or the user's face while still allowing the light source 20 to provide light to the eye of the user. The light source 20 can be one or more light emitting diodes (LEDs). However, the light source 20 can be any light delivery mechanism able to emit one or more wavelengths of light. As an example, the light source 20 can be an LED configured to emit one or more wavelengths of light. The LED can be configured to emit white light (e.g., including RGB channels) or specific wavelength(s) of light such as ultraviolet or red wavelengths (e.g., the wavelengths can be from 370-400 nm or from 640-690 nm).

When the light source 20 can provide light therapy to the eye based on an estimate of the distance between the light source and the user's eye and/or the (e.g., an estimate of where the entrance aperture of the eye can reasonably be expected to be during use of the light therapy device 10). The positioning of the light source 20 can be moved at least in part by moving and/or bending the flexible neck portion 14. The head portion 12 can also include one or more motors or actuators (not shown) for changing the orientation of the light source 20. The estimate of the distance from light source 20 and the user's eye can be larger than for a comparable bespoke pair of light therapy glasses to compensate for differences in the way different frames can sit on the user's face. The flexible neck portion 14 can move and/or bend in one or more directions (indicated by the double headed arrow) such that the head portion 12 and/or light source 20 can be moved to direct the one or more doses of light therapy into the user's eye so that the light can be directed toward the eye while keeping the body portion 16 in place. The flexible neck portion 14 can include at least one shape memory component that can be configured to hold the light source in an orientation/move the light source to a new orientation. In some instances, the shape memory component can include a spring that can be a metal spring soldered to a substrate (e.g., a copper or gold coated steel spring that can be soldered to contacts mounted on rigid portions of the substrate of the light therapy device 10).

The body portion 16 can include electronics and circuitry on at least one circuit board (or portion of a circuit board)/substrate (rigid, flexible, and/or a combination of rigid and flexible) for providing the light therapy to the eye of the user. The body portion 16 can include at least a controller 22, one or more sensors 24, an antenna (not illustrated), a power source 26 and the like. Each can be connected to the flexible circuit board, for example. The controller 22 can control at least one parameter related to providing the light therapy. For example, the at least one parameter can be time related such that the controller can have the functionality of a clock. For example, the controller 22 can determine a timing (e.g., duration, frequency, etc.) of therapy. The controller 22 can include a non-transitory memory and processor capabilities, like a microprocessor). For example, the at least one parameter related to the light therapy can include a prescribed duration of therapy, a consistent time of day for the therapy to be applied, a frequency of modulation of the therapy (e.g., a wavelength, an intensity, a time, etc.), or the like. The frequency can be in excess of a flicker fusion frequency, can correspond to an existing biological rhythm (e.g., between 25 and 60 Hz), be related to a pulse width modulation of an average power, or the like. The controller 22 can provide prescheduled and predetermined light therapy (e.g., dosages and timings pre-programmed by a health care professional). As another example, the controller 22 can include the capabilities of a non-transitory memory and a processor (e.g., at least a microprocessor) and can receive and process inputs (e.g., from at least one sensor 24, a user input, or the like) to determine when to deliver the light therapy, how to alter one or more parameters of the light therapy based on the environment, or the like.

The at least one sensor 24 can detect a variable/parameter related to the wearing of the device, the light therapy, and/or the environment of the user and to communicate the input (e.g., the variable/parameter) to the controller 22. The variable/parameter can be related to whether the user is wearing the device, the amount of ambient light (related to the environment), and/or the light therapy already delivered. The at least one sensor 24 can be one or more sensors of the same and/or diverse types. The at least one sensor 24 can be, for example, an accelerometer, an optical sensor, or the like. The controller 22 can determine whether the device is being worn, a timing of the therapy/when the deliver the therapy, etc. based on the input. For example, if the at least one sensor 24 is an accelerometer, then the accelerometer can detect motion and/or positioning of the light therapy device 10, which the controller 22 can use to determine whether the light therapy device 10 is being worn. The controller 22 can determine if light therapy should be provided based on if the light therapy device 10 is being worn (e.g., only provide light therapy if the light therapy device is being worn). As another example, the at least one sensor can be an optical sensor that can sense at least one parameter related to the amount of ambient light in the environment. The controller 22 determines when to deliver the therapy, the amount of therapy received by the user, or the like based on the amount, wavelength composition, etc. of ambient light. The input from the at least one sensor 24 can also be used to enable an entertainment function or a practical, non-therapeutic function of/related to the light therapy device 10.

Further to the at least one sensor 24 being an accelerometer, the accelerometer can be used to sense the environment in multiple ways. First, inclusion of an accelerometer can allow the light therapy device 10 to determine (via the controller 22) if the light therapy device is being worn and when to deliver the light therapy based on acceleration cues. Such acceleration cues can be passive on the part of the user or active. For example, the light therapy device 10 may be controlled (by the controller 22) to start providing light therapy (via the light source 20) only after the light therapy device has been resting for a predetermine amount of time and then been active in a manner consistent with wear for at least a second amount of time. In another example, if two light therapy devices 10 can be used (one on each arm with reference to FIG. 1) with no direct communication with each other, then the timing of light therapy can by synchronized to peaks of acceleration or correlation to a predetermined set function, in a roughly determined interval. A precise coordination of start time can give an impression of quality. In another example, the orientation of the light therapy device 10 with respect to gravity (measured by the accelerometer or other sensor) when worn can by used (by the controller 22) to identify the light therapy device as being worn and/or what side of the user's face the light therapy device is being worn on.

Passive inputs can also allow the light therapy device 10 to act as a behavior and/or health monitor in conjunction with an application on an external device (e.g., computer, smartphone, etc.) in communication with the light therapy device. For example, the light therapy device 10 (via the accelerometer (and/or other sensor) and the controller 22) can track head angle or head position variability as a surrogate for monitoring near point work or fixed visual focus. In one example, the light therapy device 10 can signal (by the controller 22 and the light source 20) the user to take a break, look up, do a head roll, or the like, if the user is observed as focusing for a period of time in excess of a predetermined health recommendation.

In a further example, the light therapy device 10 can determine (via the controller 22 with accelerometer data) when the glasses the light therapy device is attached to have been removed and pause therapy till the glasses are put back on. If the glasses (and therefore the light therapy device 10) are motionless in any orientation for a given amount of time, the controller 22 can determine the glasses are not being worn. Similarly, if the glasses (and therefore the light therapy device 10) are maintained upside down or at an extreme tilt for another period of time, then the controller 22 can determine the glasses are not being worn. This allows the light therapy device 10 to pause operations when the user sets the glasses down on a table, in a case, or in a handbag and to resume when the glasses (with the light therapy device 10 attached) are later being worn in an expected orientation.

The light therapy device 10 can have another example response to explicit user gestures (e.g., based on gestures preprogrammed in the controller 22 to mean a command). For example, the user can force the light therapy device 10 to stop or pause providing light therapy by performing a head motion (sensed by the accelerometer and/or other sensor) that is unlikely to occur in otherwise normal circumstances, such as drawing exactly two counter-clockwise circles with the glasses in the air without a significant side tilt in a period of about 2 seconds. Gestures that are even less likely may be chosen for actions that should occur once or rarely, such as activating a BLUETOOTH pairing mode. Such gestures may form the input mechanism for games or practical data communication with the device. Feedback from the games may be delivered via the therapy light source 20 or additional feedback mechanisms such as other lights, sound, or an external connected device (e.g., smart phone, smart watch, audio device, etc.).

In situations where the at least one sensor 24 is an optical sensor, the optical sensor can measure the amount of ambient light a user is exposed to at a given time. The optical sensor can also and/or alternatively measure characteristics of the ambient light such as intensity, modulation frequency, and/or color/wavelength. An optical sensor can be positioned in/on the light therapy device 20, for example, so as to not be blocked by the eyeglasses or the user's face, ear, or hair. The controller 22 of the light therapy device 10 can use the measured amount and/or characteristics of the ambient light to, for example, correlate a user's response to total exposure and/or to compensate the light therapy provided with the ambient light to reach a prescribed total exposure. The controller 22 can also delay the timing of delivery of the light therapy based on the measured amount and/or characteristics of the ambient light (e.g., when the ambient light is at a brightness that would make the light therapy less effective).

The body portion 16 of the light therapy device 10 can also include one or more antennas (not illustrated). The one or more antennas can enable communication with one or more devices, such as a computing device (e.g., desktop, laptop, tablet, smartphone, etc.), another light therapy device 10, or the like, according to a standard wireless protocol. For example, different light therapy devices (similar to light therapy device 10) can be placed on either arm of the same eyeglasses and/or one or more arms of different eyeglasses and can communicate with each other to coordinate the therapy delivery from each of the different light therapy devices. The one or more antennas can also enable data exchange with the one or more devices (e.g., for an app on a mobile device to run a social game between one or more light therapy devices 10), receiving instructions from the one or more devices, or the like.

The body portion 16 can also include a rechargeable power source (Power Source) 26. As an example, the rechargeable power source 26 can be a rechargeable battery (e.g., able to recharge magnetically, inductively, or the like). As an example, the rechargeable battery can be recharged via a magnetically attached connector and can include exposed metalized pads on the electrical connection substrate in proximity to magnets adhered to the electrical connection substrate. As another example, the rechargeable battery can include one or more inductive loop antennae capable of inductive charging. It should be understood that the power source 26 need not be rechargeable and may be replaceable.

The rechargeable power source 26 can be chosen such that the electrical power provided by the power source can sustain a therapeutic dosage of light while meeting requirements for light weight, reasonable size, charging convenience, and cost (e.g., to provide maximum power at sufficiently light weight, at a reasonable size, with ease of charging, and at minimal cost). The rechargeable battery 26, for example, can have a battery capacity less than 200 mAh (milliampere hours). As another example, the rechargeable battery 26 can have a battery capacity greater than 20 mAh. In a further example, the rechargeable battery can have a battery capacity greater than 20 mAh and less than 200 mAh. It should be understood that mAh is a measure of how much electrical charge a battery can hold, representing the amount of current that a battery can supply for one hour before the battery is fully discharged. As another example, the rechargeable power source 26 can be a rechargeable battery using LiPo (lithium polymer) battery technology that has a 40 mAh capacity in a low cost package measuring 20.0 mm×11.0 mm×3.0 mm and weighing less than 5 g. Such a rechargeable battery can enable hours of high current LED light therapy on a single working charge. The single working charge can be maintained for an extended period (e.g., days or weeks) by the light therapy device entering a low power mode (e.g., the controller 22 can determine a low power mode can be entered when the device is not being worn or no light therapy is programmed for a time) that can shut down the LED driver and a majority of computational functions of the microprocessor. As another example, Zn-Air (Zinc-air) batteries (commonly used as disposables in hearing aids) can also be a good option for rechargeable battery 26 and/or as a replaceable battery alternative.

The attachment mechanism 18 can be a part of and/or attached to the body portion 16 of the light therapy device 10 and can removably attach the light therapy device to the frame of the eyeglasses (e.g., in the desired form). In the main examples, the attachment mechanism 18 can removably attach the light therapy device 10 to an inner side of the arm of the eyeglasses frame. As an example, the light therapy device can be attached near (or at least a portion being directly next to) a temple of the user on the arm of the eyeglasses (as shown in FIG. 1). The attachment mechanism 18 can avoid contact with the hinge. As an example, the attachment mechanism 18 can include a mechanical attachment mechanism, such as one or more rubber/elastic band (e.g., substantially transparent, and/or colored), one or more spring clips, or the like. An index matching layer (with at least one index matching material) can be positioned between the one or more rubber/elastic bands and the arm of the eyeglasses frame in some examples, which has an index of refraction that closely approximates that of the frame to make the elastic/rubber bands less visible. As a further example, the attachment mechanism 18 can include an adhesive (e.g., a removeable adhesive to facilitate removal of the light therapy device 10 from the eyeglasses for transfer to another pair of eyeglasses). In another example, the attachment mechanism 18 can include a combination of one or more mechanical mechanisms and/or one or more adhesive.

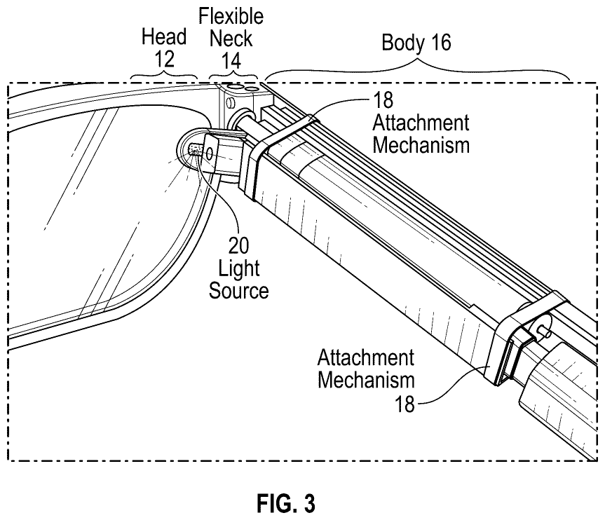

Referring now to FIG. 3, illustrated is an example of the light therapy device 10 of FIG. 1 implemented on an eyeglass frame with an external attachment mechanism 18. As noted, the light therapy device 10 has a head portion 12 (with light source 20), a flexible neck portion 14, and body portion 16. The body portion 16 includes at least a portion that can be an outer side/shell or casing of a rigid material that can hold and/or protect the interior components described in detail above. The attachment mechanism 18 is shown as two attachment mechanisms on either end of the body portion 16. The attachment mechanism 18 can be, as shown, transparent dental rubber bands (e.g., not impede the fashion of the eyeglasses) and hold the device in place on the arm. The bands (attachment mechanism 18) can be stretched to wrap around the body portion 16 of the light therapy device and the arm of the eyeglasses and then contract to hold the light therapy device in place on the arm. The bands (attachment mechanism 18) can engage attachment features (e.g., rigid portions, bump-outs, troughs, etc.) of the body portion 16 of the light therapy device. The invisibility of such bands can be increased by excluding a layer of air between the band and the arm (e.g., by including index matching gel, water, and/or adhesive). In some instances, the materials of the bands and the arm of the frames can simply be pressed together to remove the layer of air (depending on the materials). However, it should be noted that the attachment mechanism 18 may alternatively include one or more brightly colored bands that complement or contrast the colors of the glasses as desired by the user. It should also be noted that other forms of attachment mechanism 18 (e.g., one or more clips, adhesives, or the like) are possible (individually or in combination with the bands) as long as the attachment mechanism 18 attaches the light therapy device 10 to the frame.

The flexible neck portion 14 can connect the head portion 12 to the body portion 16. The head portion 12 and/or the flexible neck portion 14 can at least partially include (or be substantially made of) a substrate (or circuit board) of/with the body portion 16, where at least the flexible neck portion is a flexible portion of the substrate (or circuit board). The flexible neck portion 14 can be bent to direct the light source 20 in the head portion 12 towards the eye (and/or toward a target area in the eye). Once bent the flexible neck portion 14 can stay in that position until acted on by another force. As shown the head portion 12 can be bent away from the frame with the neck portion 14 bent in front of the hinge of the frames and may be at least partially in the peripheral view of the user such that the light source 20 is positioned substantially at the convergence of the arm of the eyeglasses the rim of the lenses of the eyeglasses.

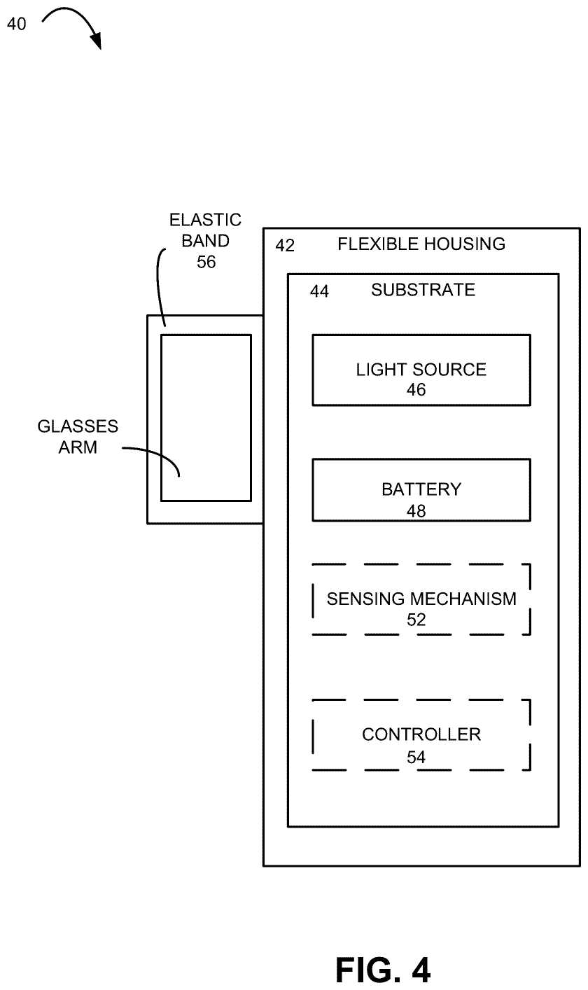

Referring now to FIG. 4, illustrated is another light therapy device 40 (similar to light therapy device 10) attachable to an inner side of an arm of an eyeglass frame proximal to a hinge of the eyeglass frame. The light therapy device 40 is shown attached to a glasses arm by an elastic band 56 (similar to attachment mechanism 18) (e.g., the elastic band is stretched over the arm of the eyeglasses frame and contracts to hold the light therapy device in place). The light therapy device 40 can include a flexible housing 42 that interfaces with or is contiguous with the elastic band 56 (e.g., the attachment mechanism) that attaches the light therapy device to the eyeglasses frame. The flexibly housing 42 can at least partially encapsulate at least a portion of a substrate 44 (e.g., an electrical connection substrate and/or circuit board) and at least some of the other components of the light therapy device 40. The other components of the light therapy device 40 can include aa light source 46 (similar to light source 20), a battery 48 (similar to power source 26), and additional components like sensing mechanism 52 (similar to sensor(s) 24), controller 54 (similar to controller 22), an antenna (not illustrated), and the like coupled to the substrate 44. The substrate 44 can have at least one flexible section and may have at least one rigid section. For example, the substrate 44 can include a hybrid rigid-flex printed circuit board (PCB). The flexible housing 42 can hold at least one of the flexible portions of the substrate 44 around the battery 48 and aim the light source 46 toward the eye (and/or help the substrate aim the light source toward the eye).

The hybrid rigid-flex PCB can include an epoxy impregnated fiberglass board and a multilayer flexible PCB with active electrical contacts. The hybrid rigid-flex PCB can be created, for example, by adhering the epoxy impregnated fiberglass board to the multilayer flexible PCB that includes active electrical contacts. Rigid sections of the hybrid rigid-flex PCB can provide mechanical stability (e.g., for soldered electrical connections), while flexible sections of the hybrid rigid-flex PCB can be similar to the flexible neck 14 described above and/or can wrap around portions of itself in a burrito like fashion. For example, the substrate 44 can include at least one flexible section (similar to the flexible neck 14) that can be bent to position the light source 46 toward the eye (and/or a target area such as the pupil). At least one flexible portion of substrate 44 can also form a tube around the battery 48 and at least some of the other components (e.g., sensing mechanism 52, controller 54, etc.). The hybrid structure can provide for a conformal packing of the required components with a minimum number of connectors. For example, the widest cross-section of the light therapy device 40 can be approximately 10 by 25 mm. The hybrid rigid-flex PCB can allow the active area for component connection to be bent out of a single plane and doubled up to achieve more circuit area to allow for more components without requiring a stack of multiple boards connected by risers or other interconnects. The flexible part of the substrate 44 (e.g., the hybrid rigid-flex PCB) can be wrapped like a burrito around the battery with the majority of components (e.g., other than the light source 46 and in some instances the sensing mechanism 52) facing inward. This “burrito” can further be filled with an epoxy encapsulation compound to provide high mechanical stability and electrical isolation of components from moisture and dust ingress. The flexible housing 42 may also aid in the isolation from moisture and dust).

A flexible portion of the substrate 44 can position the light source 46 to float (or appear to float from the use's visual perspective) near the hinge of the eyeglasses and can be sufficiently compliant to allow the arm of the glasses to bend closed. The flexible portion of the substrate 44 can also include a shape memory component that can be adhered to return the light source 46 to an orientation after the light source 46 has been moved from the orientation (e.g., when the glasses arms are opened, the light the light source can return to its previous position angled towards the eye). A spring wire, for example, can supports the light source 46 and help the flexible portion of the substrate 44 (which is like the neck portion 14 above) return to a usable position after the flexible portion of the substrate is bent beyond the elastic range of the flexible circuit board substrate. The spring can be soldered to the substrate 44. For example, the spring can be made of a copper or gold coated steel spring to make the spring easier to solder to contacts mounted on rigid portions of the substrate 44. Additionally, features formed directly into the one or more rigid portions of the substrate 44 may provide mounting hooks onto which elastic bands may be attached to fix the devices to the arm(s) of the eyeglasses.

The light therapy device 40 can also include a sensing mechanism 52 and a controller 54 coupled to the substrate 44. The sensing mechanism 52 can detect a property of an environment of the device (e.g., amount, intensity, wavelength, etc. of ambient light, acceleration or positioning relative to the environment, or the like). The controller 54 can be coupled to the sensing mechanism 54 and can include at least a non-transitory memory and a processor (e.g., microprocessor) with instructions to determine whether the light therapy device 40 is being worn based on the property of the environment of the device. The controller 52 can include and execute a number of other instructions as well. For example, the controller 52 can be coupled to the light source 46 and can control one or more parameters of the light source (e.g., in response to a predetermined automatic schedule, a user input, an input from the sensing mechanism 54, or the like). The controller 52 can, in some instances, control the light source 46 for entertainment functions and/or non/therapeutic functions (e.g., games, socialization, exercising, behavioral studies, or the like) in response to a user input.

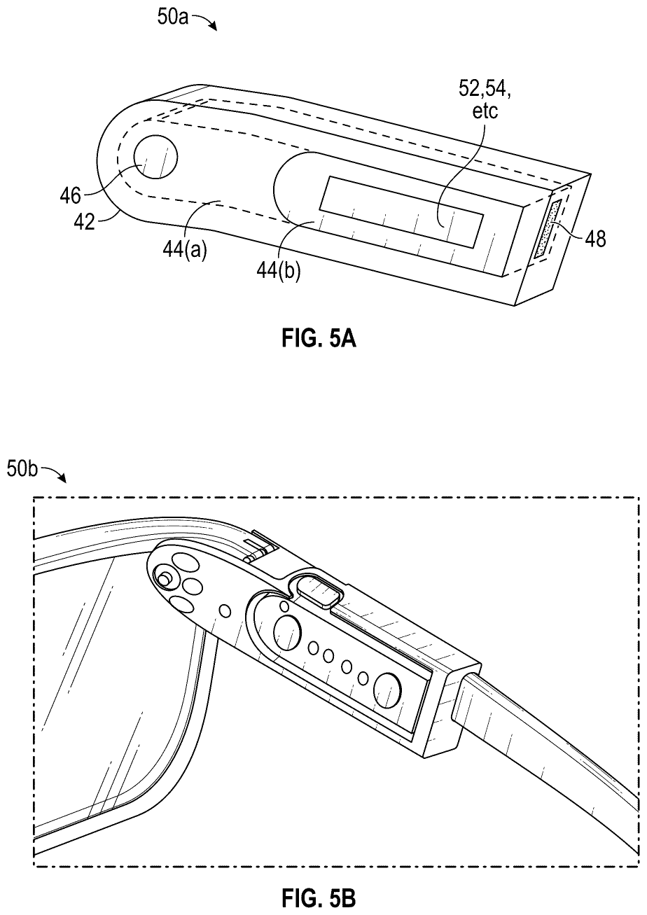

FIG. 5A shows an example diagram 50a and FIG. 5B shows an image 50b of the light therapy device 40 of FIG. 4. In the example diagram of FIG. 5A, the numbering is the same as in FIG. 4 except that a flexible “neck” portion of the substrate is represented as 44a and the rigid part of the substrate is represented as 44b. The battery 48 may be at least partially encapsuled in a burrito of substrate 44 represented by the dotted lines that can be within the flexible housing 42. The flexible housing 42 can include one or more windows to at least a portion of the substrate (e.g., a rigid portion of the substrate 44 (b)) to expose one or more control, indicator, sensing, or power components of the light therapy device 40. For example, an end of the flexible housing 42 can include an opening for connecting the battery 48 with a charger and/or for allowing a wired data communication connection (not shown). Another window in the flexible housing 42 can have the light source 46 protruding from the housing to emit light into an eye of the user wearing the eyeglasses frame with the device. The image 50b shows an example implementation of the diagram 50a used on a pair of eyeglasses. In the image 50b the elastic band is continuous with the housing and is stretched over the arm of the eyeglasses. The housing in image 50b is an insert molded silicone jacket material that can assist to aim the light source toward the eye. The device is shown as including four terminals of a USB connection (e.g., four smaller dots in a line) that can provide the ability to charge the device when it is not being worn, as well as upload and download data from the device by means of a standard charging cable (not shown) that can be held in place by thin disk magnets (larger dots flanking the USB connection terminals) connected to the surface of the substrate.

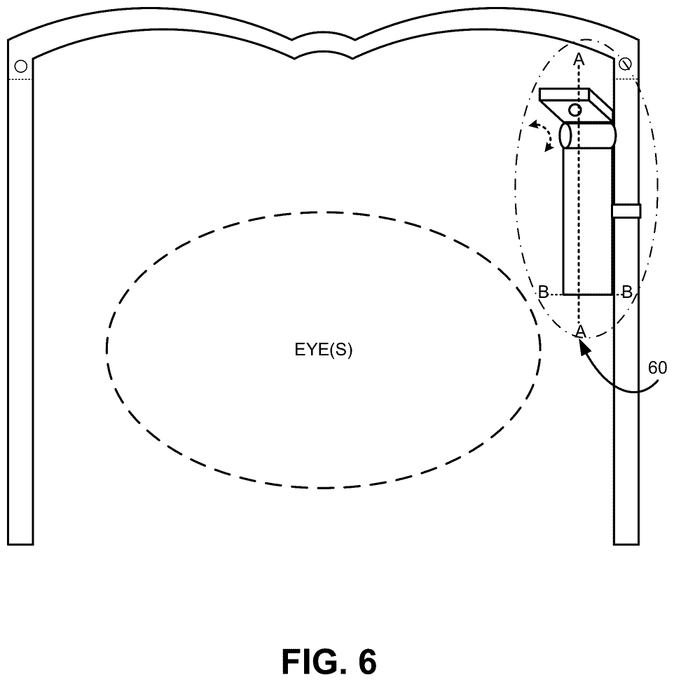

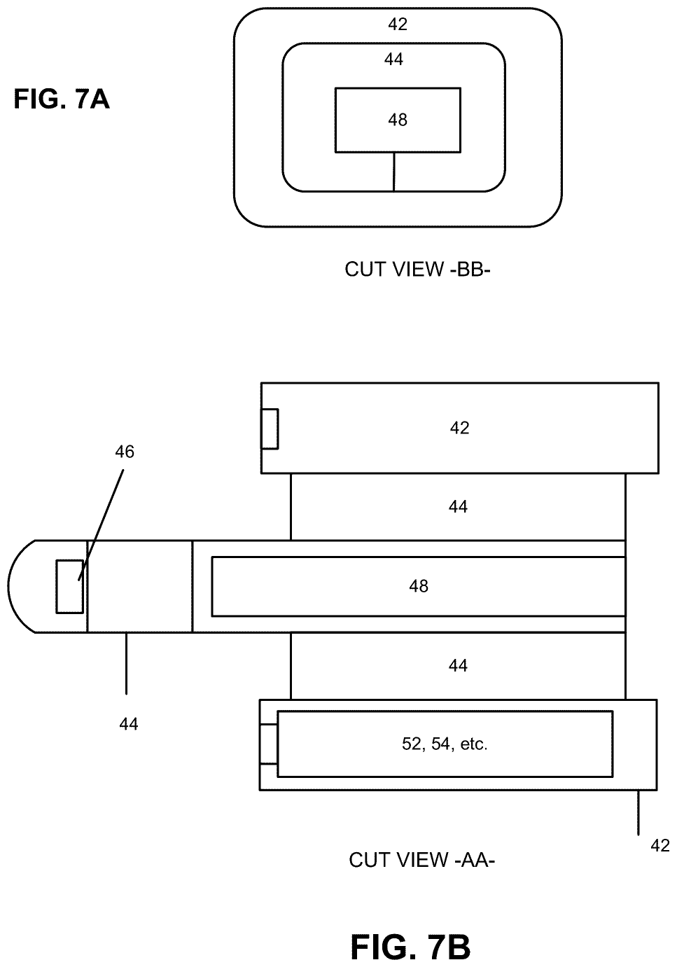

FIG. 6 shows the illustration of FIG. 1 with cut lines -BB- and -AA- through the light therapy device 60. FIG. 7A shows the cut views of the light therapy device along cut lines -BB- and while FIG. 7B shows a view along cut line -AA-, using the numbering of FIG. 4. FIG. 7A shows a posterior end view of the light therapy device 40 along cut line -BB- to show the “burrito” (generally referring to a curved/rounded substrate 44) form described above. The substrate 44 (e.g., at least one flexible section of the substrate) can be wrapped around the battery 48 in at least one direction (and other components not seen in this view). The flexible housing 42 can then encapsulate at least a portion of the substrate 44 wrapped around the battery. FIG. 7B shows a lengthwise cut view of the light therapy device along cut line -AA-. Viewed as layers, the housing 42 sandwiches the substrate 44 layers, which in turn sandwiches at least the battery 48. In this example, another portion of the substrate 44 acts as a neck holding the light source 46 away from the main body of the device. Other than the batter 48, other components can be included such as charging pads, magnets, the controller 52, a sensing mechanism 54, or the like. While not shown the housing 42 can include artwork, logos, or product labeling, or the like.

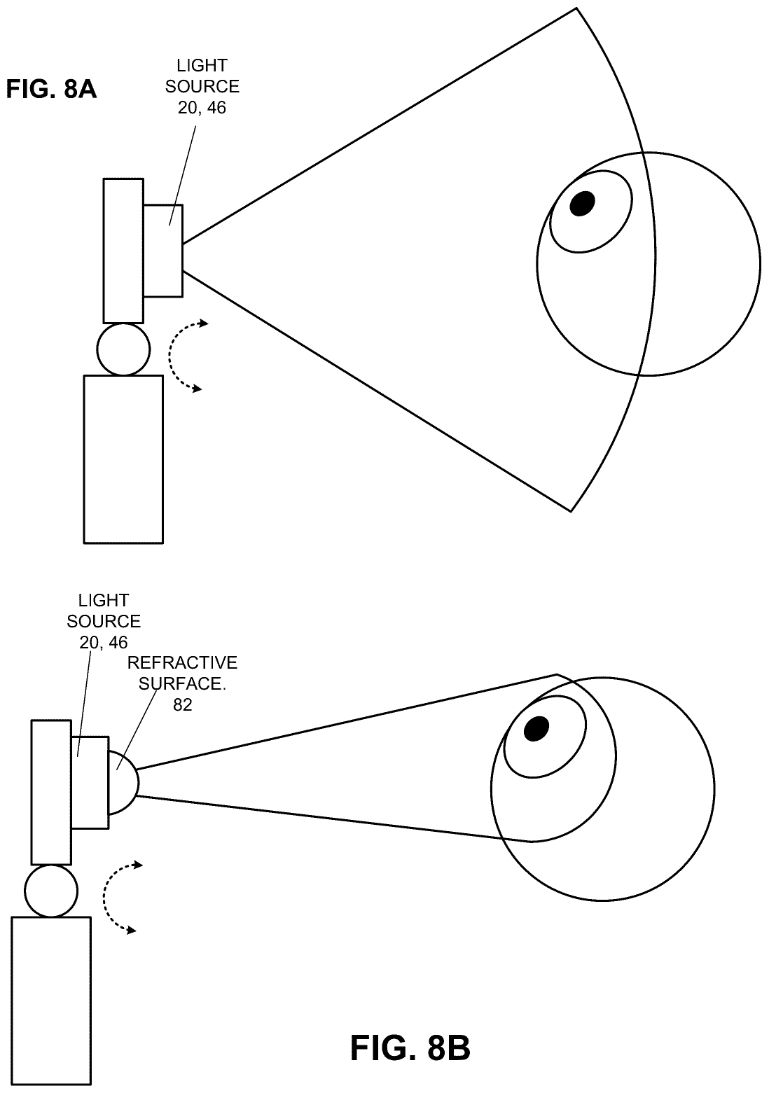

FIG. 8A and FIG. 8B show an illustration of the difference between the case in which there is no refractive surface over a light source 20, 46 of FIGS. 1 and 4, and the case in which there is a refractive surface 82 over the light source 20, 46 of FIGS. 1 and 4, respectively. FIG. 8A shows that the beam of light emitted by the light source 20, 46 toward the eye is wide and has a wide divergence angle (e.g., is generally unfocused). FIG. 8B shows a refractive surface 82 used to concentrate (focus) the light (e.g., on a pupil of the eye). As an example, the refractive surface 82 can include a lens that is adhered over the light source 20, 46 (e.g., on the portion on the light source emitting the light). As another example, the refractive surface 82 can be a substantially spherical (or semi-spherical) surface (e.g., a polymer cured in place over the light source). The substantially spherical surface can concentrate the light to a divergence angle of between 30 and 60 degrees. As a further example, the refractive surface 82 can have a lens adhered over the light source 20, 46 and a substantially spherical (or semispherical) surface.

As an example of the refractive surface 82 over the light source 20, 46, the light source can be encapsulated in a thick drop (e.g., a “glob”) of optically transparent epoxy which acts as both an electrical encapsulation and lens to concentrate the output light to a divergence angle of between 30 and 60 degrees. This example with the optically transparent epoxy cover layer reduces the required current to less than half that required by the un-lensed LED as illustrated by FIG. 8A, to achieve the same irradiance at the eye. The lens (e.g., the optically transparent epoxy drop) can be formed using a high viscosity UV curing optical cement, which in the case of a therapy involving 385 nm violet supplementation could be conveniently initiated using the therapy light source. The divergence of the LED tends to decrease as the LED surface lies nearer the focal plane of the approximately spherical surface of the drop. The thickness of the drop can be increased, and the radius of curvature decreased, by curing the optical cement in an inverted orientation such that gravity pulls the liquid drop away from the surface of the LED. Alternatively, a very precise and scratch resistant lens may be adhered to an existing LED at the same time as encapsulating the LED's electrical contacts, or a pre-lensed LED can be used if available and can be independently electrically isolated. An LED having a wider divergence (e.g., greater than 60 degrees) can be aimed only generally towards the pupil of the eye with minor change in the light delivery to the eye. As the divergence cone narrows, efficiency can be improved to the extent that the concentrated light can be aimed at the target. The LED face may be aimed toward the eye by bending the flex substrate and/or neck or by tilting the emitter towards the pupil by placing a shim underneath the LED package. The epoxy dome (e.g., refractive surface 82) can direct the light by decentering the spherical surface in the direction that the light should be aimed, which is equivalent to adding a refractive prism in series with the spherical lens. This decentering can be achieved by applying the epoxy drip or adhered lensed off-center, or by tilting the inverted package during curing of an epoxy drip.

LED spectra typically have some width and may include undesirable wavelengths. The encapsulation of the LED may also be used to modify the spectrum of the light reaching the eye by subtracting undesirable wavelengths. A coated dielectric surface, or an absorptive volume may be adhered to the LED. For example, when the light therapy utilizes LEDs with a center wavelength at 385 nm, it may be desirable to eliminate wavelengths on the short side of this spectrum which are deemed to be high energy photons beyond the therapeutic window, or to guarantee that these photons do not exist in case of manufacturing tolerances in the LED or due to temperature variations etc. Likewise fluorescent wavelengths that may commonly extend far across the visible spectrum may be attenuated to make the therapy less visible to the wearer, people in the vicinity, or to clarify the dosage lies only within the defined therapeutic window and simplify power measurement for safety verification.

### V. Methods

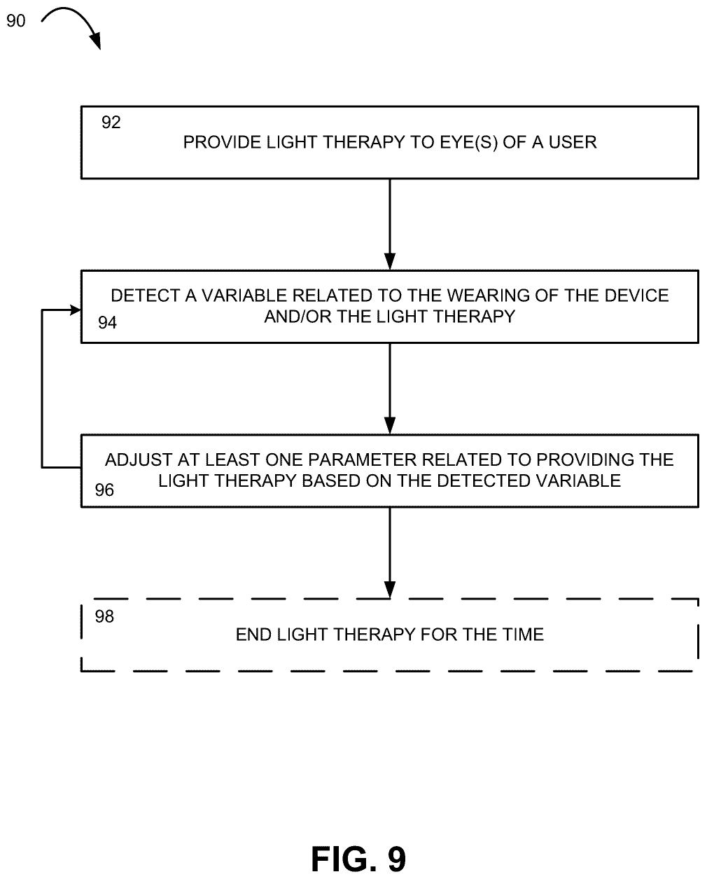

Another aspect of the present disclosure can include example methods 90-110 (shown in FIGS. 9-11) for treating one or more medical conditions using a light therapy device that is removably attachable to an arm of an eyeglass frame and fits between the arm and a user's face. Other arrangements of the light therapy device (as discussed above) are included in each of these methods 90-110. Examples of the light therapy device, and/or components of the light therapy device, are shown in FIGS. 1-8. The methods 90-110 are illustrated as process flow diagrams with flowchart illustrations that can be implemented by/with the devices and systems shown above.

For purposes of simplicity, the methods 90-110 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 90-110. It should be noted that one or more steps of the methods 90-110 can be executed by a hardware processor.

Light therapy can be delivered to a user as part of a passive process (e.g., open loop) and/or an active process (e.g., closed loop). Referring now to FIG. 9, illustrated is a method 90 for providing light therapy (e.g., using the light therapy device attached to an inner side of an arm of an eyeglass frame near a hinge of the eyeglass frame) in a closed loop, active fashion. At 92, light therapy can be provided to one or more eyes of a user of the light therapy device wearing the eyeglasses with the light therapy device attached. For example, the light therapy can include light having wavelengths from 370-400 nm, from 640-690 nm, RBG wavelengths that may be embodied in RBG channels to form white light, etc. The light therapy can be delivered according to parameters that are controlled by a controller (e.g., frequency, pulses, wavelength, duration of therapy, timing of therapy, etc.). At 94, a variable (or one or more variables) related to the wearing of the device, the environment, and/or the light therapy can be detected (e.g., by one or more sensors and sent to the controller). For example, variables can include an amount of ambient light, an intensity of ambient light, one or more wavelengths of ambient light, if the device is being worn, position of the device, acceleration of the device, etc. At 96, the at least one parameter related to providing the light therapy can be adjusted based on the detected variable. For example, the light therapy can be started/stopped, the light therapy can be postponed, the light therapy can be reduced, the frequency, the wavelength, or the duration of the light of the light therapy can be changed, or the like. Steps 94 and 96 can be repeated according to a predefined schedule. At 98, optionally, the light therapy can be ended (until the next time light therapy is scheduled to be provided to the one or more eyes of the user). The light therapy can be ended, for example, when the ambient light detected by a sensor is so bright that the light therapy would have limited/low effect or if the whole therapeutic dosage for that time has been delivered.

In an example of how sensing/communication may work, two devices (e.g., one on each arm) with no direct communication with each other may precisely coordinate a start time by synchronizing to peaks of acceleration, or correlation to an arbitrary factory set function, in a more roughly determined interval. The precise coordination of start time gives an impression of quality. The orientation of the device with respect to gravity when worn can self-identify the device as being worn on the right or left side.

As another example, passive input may also allow the device to act as a useful behavior monitor. The device may track physical activity as part of a health monitoring application. The device may track head angle or head position variability as a surrogate for monitoring near point work or fixed visual focus. The device may signal a user to take a quick break and look up and do a head roll if it observes a focused period in excess of a healthy recommendation. In a further example, the device may use accelerometer input to determine when the glasses have been removed and therefore therapy should be paused. If the glasses are motionless in any orientation, the glasses are probably not being worn. If the glasses are maintained upside down or at an extreme tilt for an extended period, the glasses are probably not being worn. This allows the user to set the glasses down on a table or in a handbag to pause therapy and resume when the glasses are later being worn in an expected orientation. The device may also respond to explicit user gestures. For example, the user may force the light therapy to stop or pause by performing a head motion that is unlikely to occur in otherwise normal circumstances, such as drawing exactly two counter-clockwise circles with the glasses in the air without a significant side tile in a period of about 2 seconds. Gestures that are even less likely may be chosen for actions that should occur once or rarely, such as activating a BLUETOOTH pairing mode. Such gestures may form the input mechanism for games or practical data communication with the device. Feedback from the games may be delivered via the therapy light sources or additional feedback mechanisms such as other lights, sound, or a different connected device.

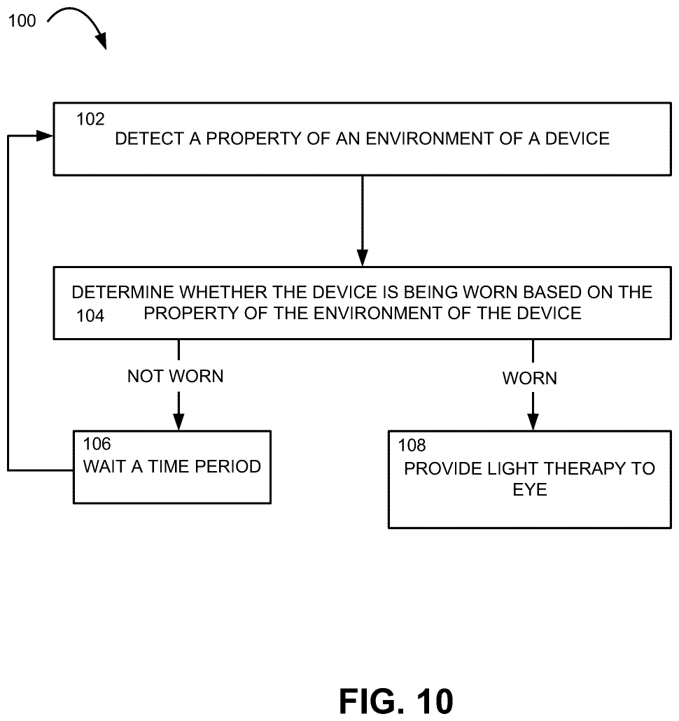

Referring now to FIG. 10, illustrated is a method 100 for determining whether to deliver light therapy to a user according to a closed loop system. It should be understood that the closed loop system is not required and an open loop system may be used to provide a simpler system. The method 100 stops light therapy from being provided if the device is not on glasses that are being worn by a user. At 102, a property of an environment of a device can be detected (e.g., sensed by an accelerometer, optical sensor, or the like). The property of the environment can be, for example, an amount of ambient light, an intensity of ambient light, one or more wavelengths of ambient light, if the device is being worn, position of the device, acceleration of the device, etc. At 104, it can be determined (e.g., by the controller) whether the device is being worn based on the property of the environment (e.g., sensed by the accelerometer, optical sensor, or the like). For example, if the device includes an accelerometer the acceleration and/or positioning of the device can be sensed and used to determine if the device is on glasses being worn by a user (or sitting in a case, in a bag, sitting on a table, or the like. If the device is not worn, at 106, a predefined time period (e.g., 5 minutes, 10 minutes, 30 can elapse before the device re-detects the property of the environment at 102. If the device is determined to be worn (e.g., on glasses worn by the user), at 108, light therapy can be provided to the eye.

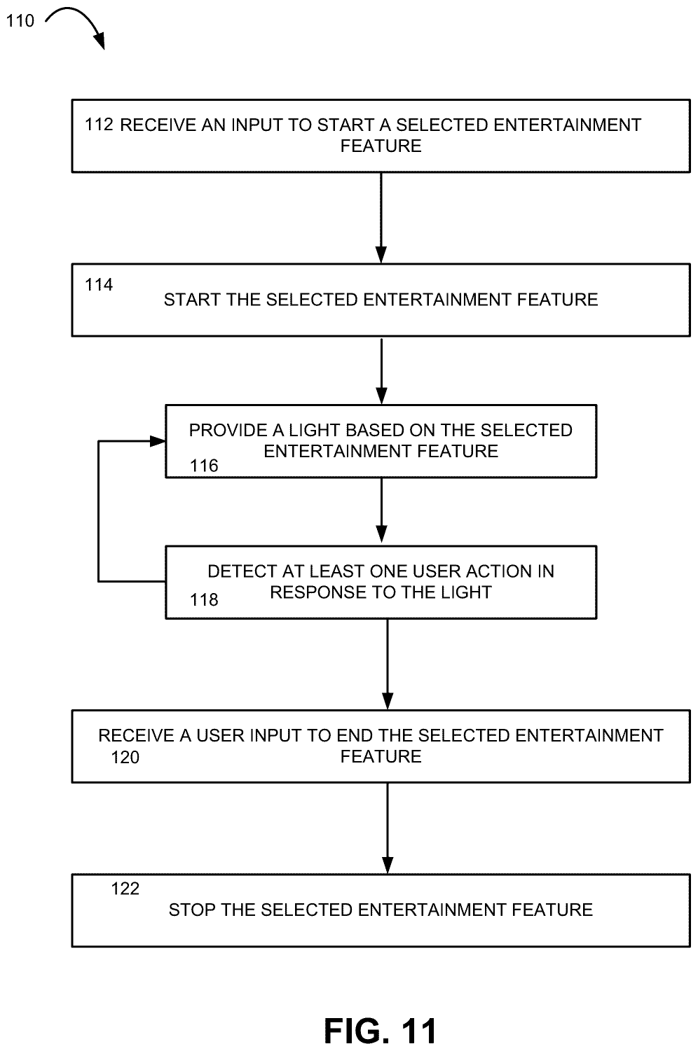

Referring now to FIG. 11, illustrated is a method 110 for providing additional functionality to incentivize a user to correctly use and not to break the light therapy device. To make it even less likely that children will use and not break the light therapy devices, some of the light therapy devices (and/or associated software) can provide additional functionality such as entertainment functions to incentivize a child to wear glasses with the device attached and receive treatments. At 112, an input can be received to start a selected entertainment feature. The input can be an input from an external device (e.g., a computer, a smartphone, or the like) in communication with the light therapy device, an input directly from the user, or the like. An input directly from a user can be, for example, a specific gesture predetermined to select a specific entertainment feature. At 114, the selected entertainment feature can be started. The entertainment feature can be, for example, a game. At 116, a light can be provided based on the selected entertainment feature. The light can be included as a portion of the light therapy dosage or can be separate from the light therapy totals. For example, the light can flash in codes or patterns (e.g., length of light flash or combination of length and colors) or can have different intensity and/or color based on a property of the environment. At 118, at least one user action can be detected in response to the light. For example, the user action can include moving the head (or another body part) in response to the light. Steps 116 and 118 can repeat to form the game until such time as the user performs an action (physical gesture and/or inputs an end command on the external device) indicating the game should end. The game can also time out if no interaction for a predetermined period of time, no ambient light for a time (e.g., shut in a case), acceleration beyond an upper limit (e.g., in moving vehicle where flashes can be dangerous) or the like. At 120, a user input can be received to end the selected entertainment feature. At 122, the selected entertainment feature can be stopped.

The entertainment features can include games, workout modes, socialization modes, or the like, for example. It should be understood that entertainment features can provide an additional value for the device which are particularly useful to prevent children from destroying the device (as some children may be less likely to destroy something that brings them amusement). Some games or modes can reward increased physical activity, promoting a healthy lifestyle overall, but not reward simple fiddling with the device or the glasses. One example game can include a memory game that can have flashes with different time durations (e.g., short, mid, and long) on left and/or right sides (and if two devices are worn) with the shortest flashes, for example, corresponding to taps by the user on an exterior of the frames, mid flashes to head nods or rotations by the user (as an example), and long flashes to sustained head tilts. Another version of a memory game can include flashing and responsive tapping or clapping in a familiar rhythm (e.g., shaving and a haircut, happy birthday, etc.). Additionally, the entertainment feature may include a social model allowing player vs player memory challenges such as pairing with a nearby device to try and beat your opponent in length of pattern matches. Another example of an entertainment feature can include a guide through three dimensions to a location by tracking rotation and distance travelled. For example, the frequency of LED blink can correspond to a level of warmness to a desired location or a pause when no change. In a social mode the device can recognize when it comes within a distance threshold of another device and can give a specific blink if previously paired (e.g., a welcome to/from a “friend”).

Other functionality beyond entertainment can include: methods for finding a phone previously paired (e.g., Bluetooth paired or the like) with the light therapy device (e.g., blinking or gaining intensity when move closer or the like), methods for controlling a phone by swiping or scrolling controls through head movements detected by the light therapy device (by an accelerometer), methods for providing a level (e.g., as a user tries to balance during a workout), or methods for working out (e.g., go up, down, left, right, faster, slower, or the like based on light commands).

New functionality including modifications to therapy, sensing methods or data recorded, gestures, and entertainment functions may be programmed on the device as a software upgrade. Such changes may be applied via wireless antennae by connecting another device such as an app on a phone, by user accessible terminals that, for example, may be applied for charging the device, or by user inaccessible terminals that, for example, may be accessible on the inside of the electrical connection substrate before the potting is complete to prevent ingress. It may be desirable to allow user software upgrades to encourage user interaction and engagement with a user-facing app throughout the customer journey. Software upgrades or settings that may only be set at the doctor's office may allow a therapy function or dosage to be set according to a prescription. Software upgrades that may be typically applied only at the factory may include lower level upgrades that cannot be confidently applied in the field or may be used so that users who must replace their device have the experience of being upgraded when they do replace their device.

From the above description, those skilled in the art will perceive improvements, changes, and modifications. Such improvements, changes and modifications are within the skill of one in the art and are intended to be covered by the appended claims.

What is claimed is:

1. A system comprising:
a head portion comprising a light source configured to provide light therapy to an eye of a user of the system;
a flexible neck portion configured to be bendable to orient the light source towards the eye; and
a body portion comprising:
a controller configured to control at least one parameter related to providing the light therapy,
at least one sensor configured to detect a variable related to the wearing of the device and/or the light therapy,
a rechargeable power source, and
an attachment mechanism configured to removably attach the system to an inner side of an arm of a glasses frame near a temple of the user.

2. The system of claim 1, wherein the flexible neck portion comprises at least one shape memory component configured to hold the light source in an orientation.

3. The system of claim 1, wherein the at least one sensor comprises an accelerometer and/or an optical sensor.

4. The system of claim 1, wherein the light source is configured to emit white light.

5. The system of claim 1, wherein the light source is configured to emit at least one of ultraviolet light with a wavelength from 370-400 nm or red light with a wavelength from 640-690 nm.

6. The system of claim 1, wherein a capacity of the rechargeable power source is greater than 20 mAh and less than 200 mAh.

7. The system of claim 1, wherein the rechargeable power source comprises a magnetically attached connector and/or an inductive loop antenna.

8. The system of claim 1, wherein the controller comprises a microprocessor configured to control at least one of a timing, a duration, or a frequency of delivery of the light therapy.

9. The system of claim 8, wherein the controller is further configured to control at least one of the timing, the duration, or the frequency of delivery of the light therapy based on the variable related to the wearing of the device and/or the light therapy.

10. The system of claim 1, wherein the attachment mechanism further comprises at least one elastic band configured to removably attach the system to an inner side of the arm of the glasses frame near the temple of the user.

11. The system of claim 1, wherein the attachment mechanism comprises at least one of a spring clip, an elastic band, or a removable adhesive.

12. A device configured to attach to an inner side of an arm of an eyeglass frame proximal to a hinge of the eyeglass frame, the device comprising:
a substrate with at least one flexible section configured to form a tube around a battery;
a light source coupled to the substrate and configured to emit light into an eye of a user wearing the eyeglass frame;
a flexible housing configured to hold the flexible section of the substrate around the battery and aim the light source toward the eye; and
an elastic band configured to attach the device to the eyeglass frame.

13. The device of claim 12, wherein the light source is coupled to a refractive surface configured to concentrate the light on a pupil of the eye.

14. The device of claim 13, wherein the refractive surface comprises a lens that is adhered over the light source.

15. The device of claim 13, wherein the refractive surface comprises a substantially spherical surface.

16. The device of claim 15, wherein the substantially spherical surface comprises a polymer cured in place over the light source.

17. The device of claim 15, wherein the substantially spherical surface concentrates the light to a divergence angle of between 30 and 60 degrees.

18. The device of claim 12, further comprising:
a sensing mechanism configured to detect a property of an environment of the device, and
a controller coupled to the sensing mechanism, wherein the controller is configured to determine whether the device is being worn based on the property of the environment of the device.

19. The device of claim 18, wherein the controller is coupled to the light source and is further configured to control the light source for entertainment functions and/or non-therapeutic functions in response to receiving a user input.

20. The device of claim 12, wherein the elastic band is configured to be stretched over the arm of the eyeglass frame and to hold the device in place on the arm.
